(19) United States
(12) Patent Application Publication
(10) Pub. No.: US 2022/0240919 A1
(43) Pub. Date: Aug. 4, 2022

(54) AUTOMATED ANCHOR INSERTION SYSTEM

(71) Applicant: Conmed Corporation, Utica, NY (US)

(72) Inventors: Alan Hernandez, Odessa, FL (US); Peter Miller, Largo, FL (US); Grady Breslich, Saint Petersburg, FL (US)

(73) Assignee: Conmed Corporation, Utica, NY (US)

(21) Appl. No.: 17/621,983

(22) PCT Filed: Jul. 2, 2020
(86) PCT No.: PCT/US20/40685

§ 371 Date: Dec. 22, 2021

Related U.S. Application Data
(60) Provisional application No. 62/869,718, filed on Jul. 2, 2019.

(51) Int. Cl. A61B 17/04; A61B 17/17

(52) U.S. Cl. CPC A61B 17/0401; A61B 2017/0409; A61B 17/1717; A61B 17/0482

(57) ABSTRACT

An automated anchor inserter system for drilling a pilot hole and inserting an anchor. The system includes a body having a first end and a second end. An input shaft extends from the first end of the body and a guide tube extends from the second end of the body. The system also includes a first drive shaft recess and a second drive shaft recess within the body. A drill drive shaft is moveable within the input shaft and an inserter drive shaft is moveable within the second drive shaft recess. In a first configuration, the input shaft and the drill drive shaft move distally together through the first drive shaft recess and in a second configuration, the drill drive shaft moves proximally relative to the input shaft. In a third configuration, movement of the drill drive shaft moves the inserter drive shaft.

### CROSS-REFERENCE TO RELATED APPLICATION

The present application claims priority to and the benefit of U.S. Provisional Patent Application No. 62/869,718, filed on Jul. 2, 2019 and entitled “Automated Anchor Insertion System,” the entirety of which is incorporated herein by reference.

### BACKGROUND OF THE INVENTION

### 1. Field of the Invention

The present invention relates to a drill guide and anchor driver and, more particularly, to an automated anchor insertion system.

### 2. Description of Related Art

Many orthopedic surgical and medical procedures require the fixation of one body to another body. Such bodies may include bone, soft tissue, and prosthetics. One body can be fixed in a position relative to another using connector devices, such as screws and suture anchors (e.g., cannulated knotless suture anchors and soft all suture anchors). For example, various orthopedic surgeries require the insertion and fixation of a suture anchor within a bone.

One example of a suture anchor is a soft suture anchor, such as the Y-Knot® device. See, e.g., U.S. Pat. No. 9,826,971. Since soft anchors are commonly made entirely of suture materials, they are sometimes called “all-suture” anchors, and generally include a fibrous construct anchor body portion (or fibrous, braided or woven fabric-type structure such as a flexible web, as described in U.S. Pat. No. 9,173,652) and a suture or filament portion.

In orthopedic surgeries, prior to insertion of a suture anchor, a pilot hole is drilled into the bone. Traditionally, a standard single barrel drill guide is placed at the desired pilot hole location (i.e., desired anchor location) on the bone. Then, a drill bit attached to a power instrument is placed through the drill guide to create the pilot hole. During this process, constant attention needs to be on the guide to ensure that the guide is not moved from the previously selected location. The power instrument is then activated and the pilot hole is created with the drill bit. The drill bit is then removed and replaced with a driver (or “inserter”) pre-loaded with the suture anchor.

While maintaining the guide placement, the anchor is then inserted into the guide and inserted into the pilot hole with the driver. Thus, throughout the entire process, the user is required to alternate between the use of a drill and a driver while maintaining the position of the guide. If the position of the guide is lost, it is very difficult to find the pilot hole location. If the location is not found, a new pilot hole must be created. If the user does not notice the guide has been moved from the original pilot hole location and the anchor is inserted into the guide, the anchor is damaged. In such instances, a user will need a new anchor loaded onto the driver.

Therefore, there is a need for an automated anchor insertion system that increases user efficiency by ensuring that the anchor is inserted into the pilot hole.

Description of the Related Art Section Disclaimer: To the extent that specific patents/publications/products are discussed above in this Description of the Related Art Section or elsewhere in this disclosure, these discussions should not be taken as an admission that the discussed patents/publications/products are prior art for patent law purposes. For example, some or all of the discussed patents/publications/products may not be sufficiently early in time, may not reflect subject matter developed early enough in time and/or may not be sufficiently enabling so as to amount to prior art for patent law purposes. To the extent that specific patents/publications/products are discussed above in this Description of the Related Art Section and/or throughout the application, the descriptions/disclosures of which are all hereby incorporated by reference into this document in their respective entirety(ies).

### BRIEF SUMMARY OF THE INVENTION

Embodiments of the present invention are directed to an automated anchor insertion system. According to one aspect, the system includes a body having a first end and a second end. An input shaft extends from the first end of the body and a guide tube extends from the second end of the body. The system also includes a first drive shaft recess within the body. A drill drive shaft is moveable within the input shaft. In a first configuration, the input shaft and the drill drive shaft move distally together through the first drive shaft recess and in a second configuration, the drill drive shaft moves proximally relative to the input shaft.

According to another aspect, the system includes a body having a first end and a second end. An input shaft extends from the first end of the body and a guide tube extends from the second end of the body. The system also includes a first drive shaft recess and a second drive shaft recess within the body. A drill drive shaft is moveable within the input shaft and an inserter drive shaft is moveable within the second drive shaft recess. In a first configuration, the input shaft and the drill drive shaft move distally together through the first drive shaft recess and in a second configuration, the drill drive shaft moves proximally relative to the input shaft. In a third configuration, movement of the drill drive shaft moves the inserter drive shaft.

According to yet another aspect, the present invention is a method for creating a pilot hole and inserting an anchor. The method includes the steps of: (i) providing a body having a first end and a second end, an input shaft extending from the first end of the body and a guide tube extending from the second end of the body, a first drive shaft recess and a second drive shaft recess within the body, a drill drive shaft moveable within the input shaft and connected to a drill bit, and an inserter drive shaft moveable within the second drive shaft recess and connected to an anchor driver; (ii) driving the input shaft, which drives the input shaft and the drill drive shaft together in a distal direction and extends the drill bit through the guide tube, drilling the pilot hole; (iii) connecting the input shaft to the first drive shaft recess; and (iv) retracting the drill bit by driving the input shaft and moving the drill drive shaft independently in a proximal direction.

These and other aspects of the invention will be apparent from and elucidated with reference to the embodiment(s) described hereinafter.

### BRIEF DESCRIPTION OF THE SEVERAL VIEWS OF THE DRAWING(S)

The present invention will be more fully understood and appreciated by reading the following Detailed Description in conjunction with the accompanying drawings. The accompanying drawings illustrate only typical embodiments of the disclosed subject matter and are therefore not to be considered limiting of its scope, for the disclosed subject matter may admit to other equally effective embodiments. Reference is now made briefly to the accompanying drawings, in which:

FIG. 1 is a perspective view schematic representation of an automated anchor insertion system, according to an embodiment;

FIG. 2 is an exploded view schematic representation of the automated anchor insertion system, according to an embodiment;

FIG. 3 is a partial sectional view schematic representation of the automated anchor insertion system, according to an embodiment;

FIG. 4 is a partial cross-sectional front view schematic representation of the anchor system in a start configuration, according to an embodiment;

FIG. 5 is a partial cross-sectional front view schematic representation of the anchor system in a first drilling configuration, according to an embodiment;

FIG. 6 is a partial cross-sectional front view schematic representation of the anchor system in a second drilling configuration, according to an embodiment;

FIG. 7 is a partial cross-sectional front view schematic representation of the anchor system in a first retracted configuration, according to an embodiment;

FIG. 8 is a partial cross-sectional front view schematic representation of the anchor system in a second retracted configuration, according to an embodiment;

FIG. 9 is a partial cross-sectional front view schematic representation of the anchor system in a first insertion configuration, according to an embodiment; and

FIG. 10 is a partial cross-sectional front view schematic representation of the anchor system in a second insertion configuration, according to an embodiment.

### DETAILED DESCRIPTION OF THE INVENTION

Aspects of the present invention and certain features, advantages, and details thereof, are explained more fully below with reference to the non-limiting examples illustrated in the accompanying drawings. Descriptions of well-known structures are omitted so as not to unnecessarily obscure the invention in detail. It should be understood, however, that the detailed description and the specific non-limiting examples, while indicating aspects of the invention, are given by way of illustration only, and are not by way of limitation. Various substitutions, modifications, additions, and/or arrangements, within the spirit and/or scope of the underlying inventive concepts will be apparent to those skilled in the art from this disclosure.

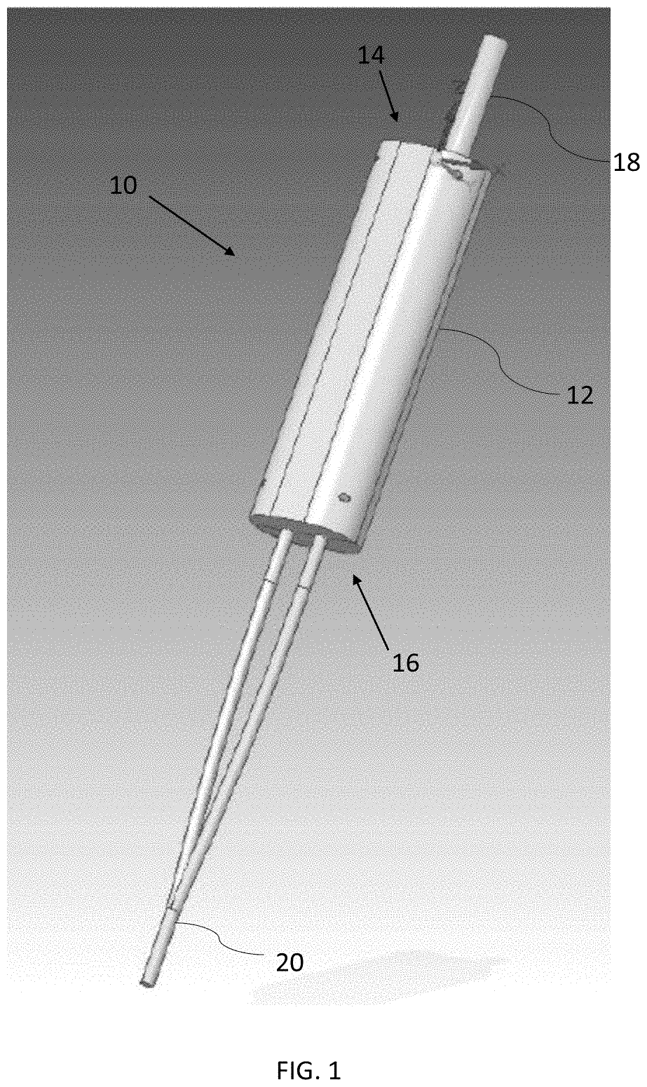

Referring now to the figures, wherein like reference numerals refer to like parts throughout, FIG. 1 shows a perspective view schematic representation of an automated anchor insertion system 10 (also referred to as “anchor system” or “drill system”), according to an embodiment. The anchor system 10 includes a body 12 with a first end 14 and a second end 16. An input shaft 18 extends proximally from the first end 14 and a guide tube 20 extends distally from the second end 16, as shown in FIG. 1.

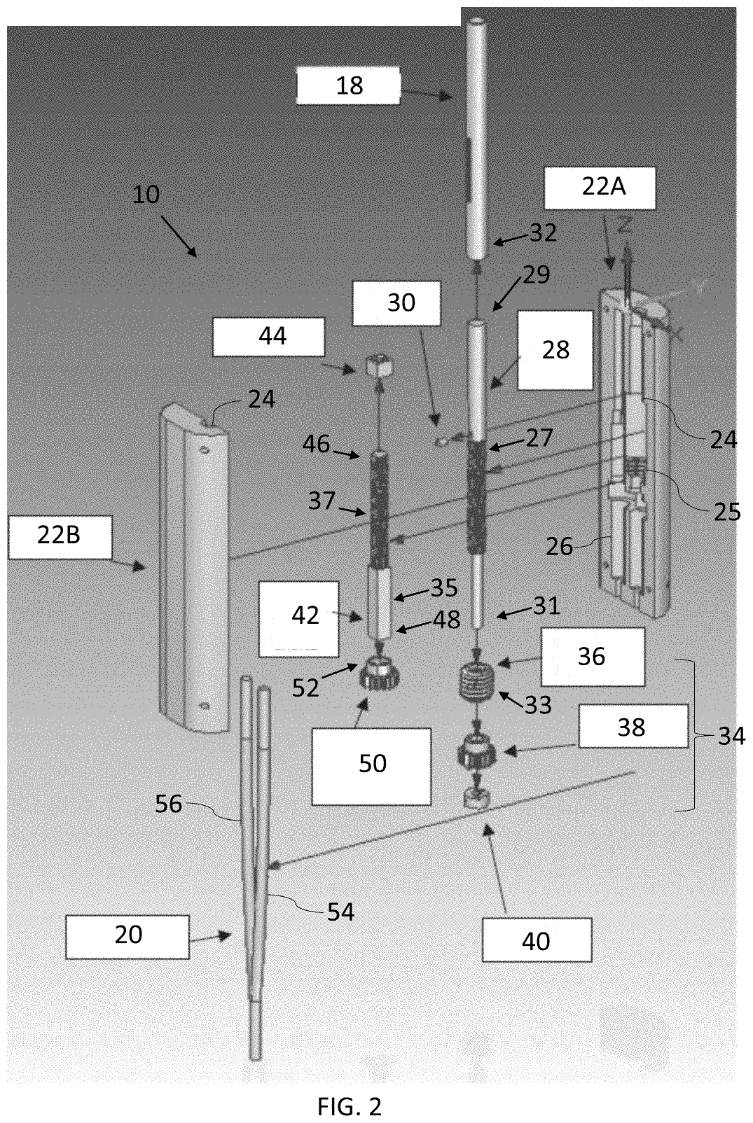

Referring now to FIG. 2, there is shown an exploded view schematic representation of the automated anchor insertion system 10, according to an embodiment. In the embodiment shown in FIG. 2, the body 12 of the anchor system 10 is composed of two pieces, a first outer body portion 22A and a second outer body portion 22B. It is contemplated that the body 12 can be machined as a single component in an alternative embodiment. In FIG. 2, the first and second outer body portions 22A, 22B each comprise a first drive shaft recess 24 and a second drive shaft recess 26. When the first and second outer body portion 22A, 22B are connected or otherwise joined, the first drive shaft recesses 24 form a first drive shaft channel and the second drive shaft recesses 26 form a second drive shaft channel.

As shown in FIG. 2, the first drive shaft recesses 24 (and first drive shaft channel) extend through the first end 14 of the body 12 (including the first and second outer body portions 22A, 22B). In the depicted embodiment, the first drive shaft recesses 24 include a threaded portion, i.e., threads 25 machined into the first drive shaft recesses 24. The first drive shaft recesses 24 are sized and configured to receive the input shaft 18 and a drill drive shaft 28. As shown in FIG. 2, the input shaft 18 is positioned proximal relative to the drill drive shaft 28. The drill drive shaft 28 comprises a threaded portion with threads 27 between its proximal end 29 and distal end 31.

Central to the functionality of the anchor system 10 is a drill pin 30, which is fixed to and extends from the drill drive shaft 28. A distal end 32 of the input shaft 18 is attached to a drill retract gear 36 (also referred to as “collar”). The drill retract gear 36 has external threads 33 sized and configured to engage with and couple to the threads 25 of the first drive shaft recesses 24. The drill retract gear 36 also has internal threads (not shown) configured to engage with and couple to the threads 27 of the drill drive shaft 28. The drill drive shaft 28 extends through one or more gears 34, including the drill retract gear 36. The distal end 31 of the drill drive shaft 28 extends through the drill retract gear 36 and a spur gear 38 and attaches to a dog gear 40. Thus, the dog gear 40 moves with the drill drive shaft 28.

Still referring to FIG. 2, the second drive shaft recesses 26 are sized and configured to receive an inserter drive shaft 42. The inserter drive shaft 42 has a distal square portion 35 (i.e., a portion that has a square cross-section) and a proximal threaded portion (with threads 37). A square nut (or hub) 44 is fixed within the second drive shaft recess 26 and a proximal end 46 of the inserter drive shaft 42 is rotated through the square nut 44, allowing the square nut 44 to engage and grab the threads 37 of the proximal threaded portion. A distal end 48 of the inserter drive shaft 42 is extendable or otherwise moveable through a spur gear 50. In particular, the spur gear 50 has a square drive 52 to accommodate the distal square portion 35 of the inserter drive shaft 42, as shown in FIG. 2. The square nut 44, inserter drive shaft 42, and spur gear 50 are within the second drive shaft recesses 26 (and second drive shaft channel).

As stated above and shown in FIG. 2, the anchor system 10 has a guide tube 20 extending from the second end 16 of the body 12. In particular, the guide tube 20 is bifurcated such that the guide tube 20 splits into a first guide tube 54 and a second guide tube 56. In the depicted embodiment, the first guide tube 54 extends into the first drive shaft recesses 24 and the second guide tube 56 extends into the second drive shaft recesses 26. In particular, the first guide tube 54 is fixed to the first drive shaft recesses 24 and the second guide tube 56 is fixed to the second drive shaft recesses 26.

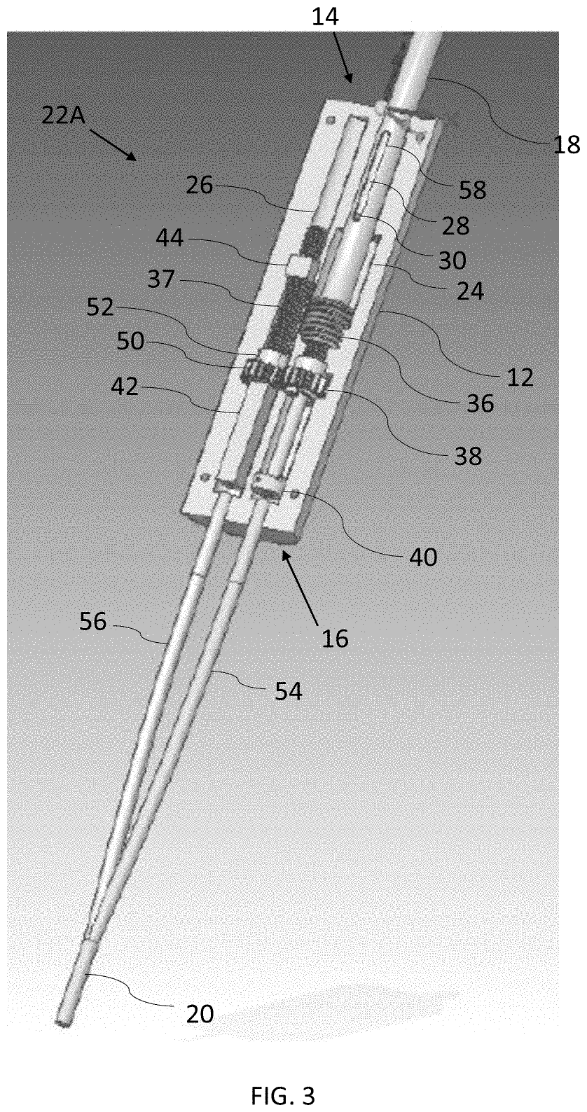

Turning now to FIG. 3, there is shown a partial sectional view schematic representation of the automated anchor insertion system 10, according to an embodiment. In the partial sectional view, the first outer body portion 22A is shown; however, the second outer body portion 22B looks like a mirror image of FIG. 3. In the depicted embodiment, the input shaft 18 is within the first drive shaft recess 24. As shown in FIG. 3, the input shaft 18 is cannulated such that input shaft 18 extends over the drill drive shaft 28. In other words, drill drive shaft 28 is sized and configured to fit within the input shaft 18. As shown in FIG. 3, the input shaft 18 extends over the drill drive shaft 28 to the drill retract gear 36, while the drill drive shaft 28 extends through the drill retract gear 36 and the spur gear 38 and connects to the dog gear 40.

Still referring to FIG. 3, the inserter drive shaft 42 is within the second drive shaft recess 26. The square nut 44 is shown engaging the threads 37 the inserter drive shaft 42. The inserter drive shaft 42 is extended through the square drive 52 of the spur gear 50. The spur gear 50 of the inserter drive shaft 42 is adjacent to and can sometimes be engaged with the spur gear 38 of the drill drive shaft 28, as described in detail below. As shown in FIG. 3, the drill pin 30 attached to the drill drive shaft 28 is shown extending through a pin guide 58 of the input shaft 18 for providing indications during the surgical procedure, as described in detail below. The pin guide 58 has additional functions, such as transmitting the torque between the input shaft 18 and the drill drive shaft 28.

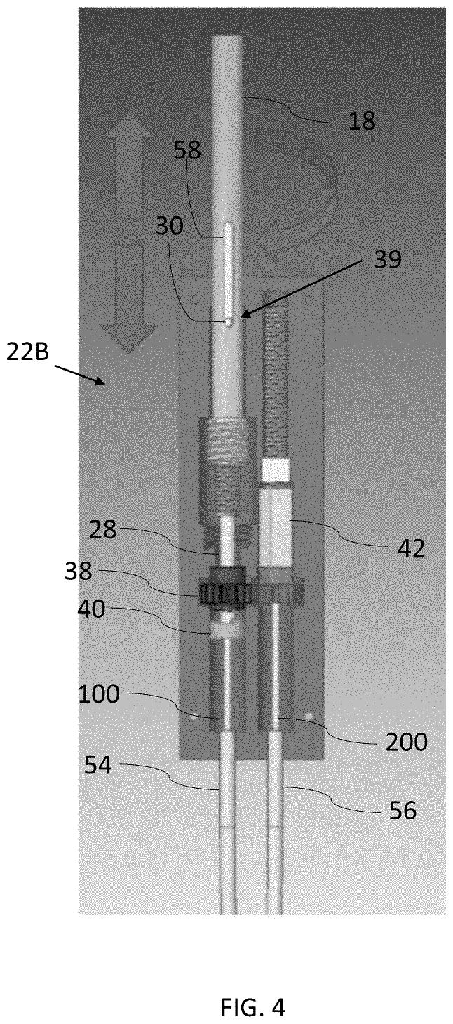

Turning now to FIGS. 4-10, there are shown partial cross-sectional front views schematic representations of the automated anchor insertion system 10 at various stages of use, according to an embodiment. In the depicted embodiment, a drill bit 100 is shown attached to and extending from the drill drive shaft 28. The drill bit 100 extends from the drill drive shaft 28 and through the first guide tube 54. As also shown in FIGS. 4-10, an anchor driver (or inserter) 200 is shown attached to and extending from the inserter drive shaft 42. The anchor driver 200 extends from the inserter drive shaft 42 and through the second guide tube 56. In the depicted embodiment, the second outer body portion 22B is shown; however, the first outer body portion 22A looks like a mirror image of those shown.

FIG. 4 shows the anchor system 10 in a start configuration, according to an embodiment. In the start configuration, the anchor system 10 is placed at the desired location at the surgical site and the user has full control of the translation and rotation of the drill system 10. As shown, the input shaft 18 and the drill drive shaft 28 are in an extended state which is held in this state by threaded collar 36 (via engagement with and coupling to the threads 27 of the drill drive shaft 28) such that the drill pin 30 is distal (or at a distal end 39) of the pin guide 58. In the start configuration, the dog gear 40 is spaced from or otherwise not engaged with the spur gear 38.

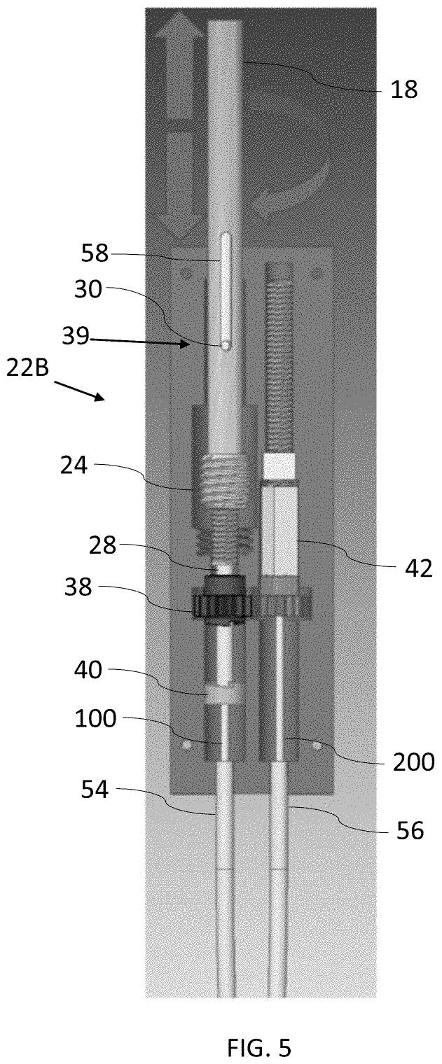

FIG. 5 shows the anchor system 10 in a first drilling configuration, according to an embodiment. From the start configuration, the input shaft 18 begins to drives the drill drive shaft 28, advancing the drill bit 100 through the first guide tube 54. The user pushes down on the input shall 18, driving the drill drive shaft 28. Pressure can be applied by the user to the input shaft 18 in the distal direction through a handpiece or other power device (not shown) connected to the input shaft 18. As the input shaft 18 drives the drill drive shaft 28, they both move distally while rotating within the first drive shaft recess 24. The drill retract gear 36 rotates with both the input shaft 18 and the drill drive shaft 28. As shown in FIG. 5, the drill pin 30 remains distal (or at a distal end 39) of the pin guide 58. In the first drilling configuration, the dog gear 40 is moved farther distally from the spur gear 38 due to distal translation of the drill drive shaft 28. In an embodiment, in the first drilling configuration (FIG. 5), the drill bit 100 is approximately halfway through the action of drilling into a media (i.e., halfway through creation of the pilot hole).

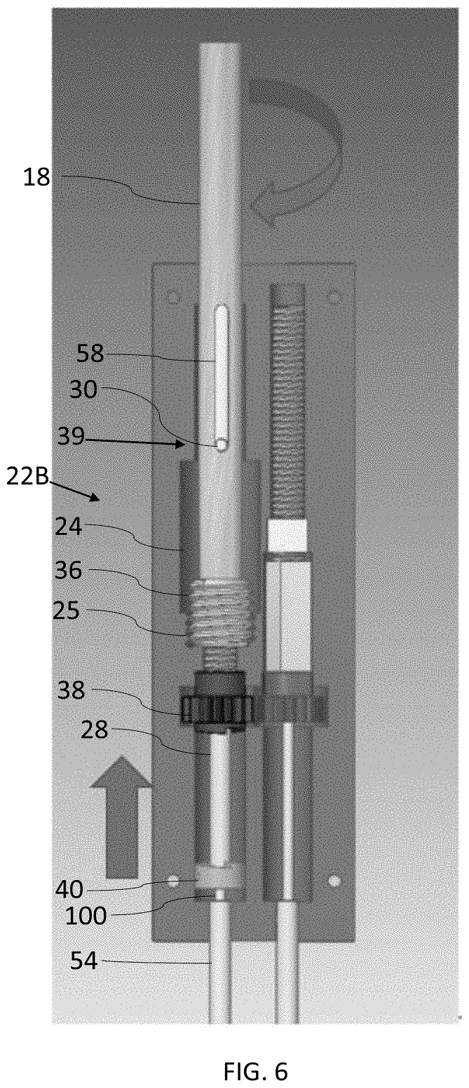

From the first drilling configuration, the user continues to push apply force in the distal direction) the input shaft 18, which continues to drive the drill drive shaft 28 and advance the drill bit 100 through the first guide tube 54 to a desired depth for pilot hole creation. The input shaft 18 and the drill drive shaft 28 advance until they bottom out at the second drilling configuration, as shown in FIG. 6. At the second drilling configuration, the threaded drill retract gear 36 rotates into the internal threads 25 of the first drive shaft recess 24. Once the threads 33 of the drill retract gear 36 engage the internal threads 25 of the first drive shaft recess 24, the drill retract gear 36 becomes fixed relative to the body 12. As shown, in the second drilling configuration, the dog gear 40 is farther distally from the spur gear 38 than it is in the first drilling configuration and the drill pin 30 remains distal (or at a distal end 39) in the pin guide 58. Once the desired depth for pilot hole creation has been reached (in the second drilling configuration), the user no longer has control of the translation due to the fixation of the drill retract gear 36 to the body 12.

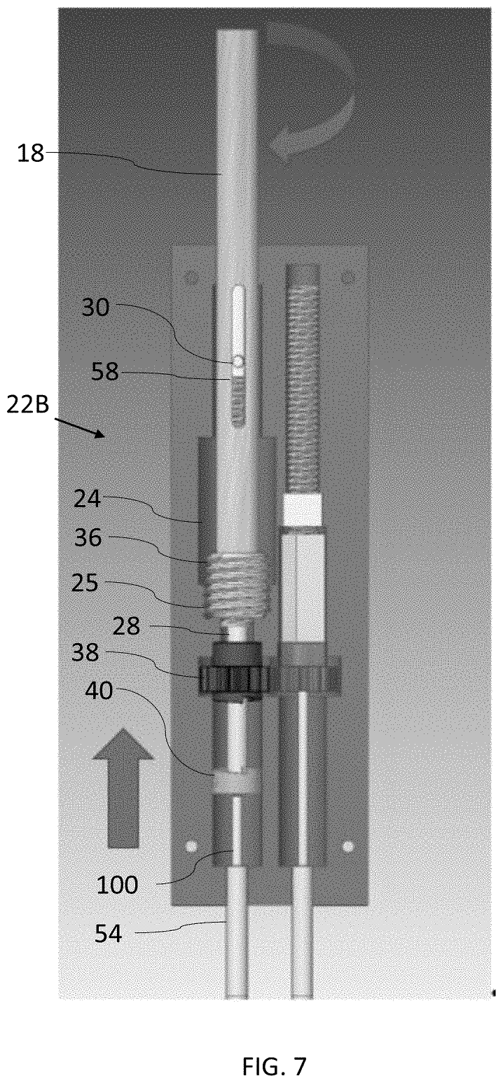

From the second drilling configuration, the user must then continue applying the rotational input (to the input shaft 18) to complete the insertion process. The anchor system 10 will start to retract the drill bit 100 via the same rotational input that was used to drive it distally, which simplifies the anchor system 10 and removes dependency of the user having to perform the sequence of tasks correctly. Specifically, the threads 27 of the drill drive shaft 28 rotate relative to the internal threads (not shown) of the now fixed drill retract gear 36, which pulls the drill drive shaft 28 proximally into the input shaft 18, thereby pulling the drill bit 100 out of the pilot hole. In FIG. 7, the anchor system 10 is in a first retracted configuration, according to an embodiment. In the first retracted configuration, the drill drive shaft 28 has moved proximally while the input shaft 18 remains in place and the threaded drill retract gear 36 remains in the internal threads 25 of the first drive shaft recess 24, The proximal retraction of the drill drive shaft 28 is shown via the position of the drill pin 30 in the pin guide 58 of the input shaft 18. The drill pin 30 has moved proximally within the pin guide 58 as compared to its positioning in the start configuration and the first and second drilling configurations. In the first retracted configuration, the dog gear 40 has moved proximally, closer to the spur gear 38, as compared to its position in the second drilling configuration.

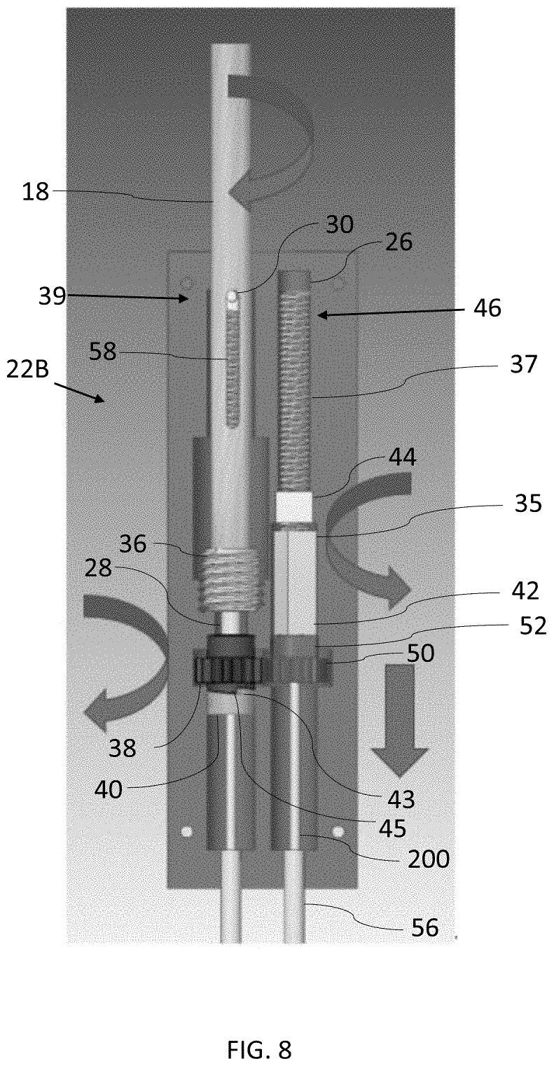

FIG. 8 shows the anchor system 10 in a second retracted configuration, according to an embodiment, From the first retracted configuration, the drill drive shaft 28 moves farther in the proximal direction until it is fully retracted in the second retracted configuration. The full proximal retraction of the drill drive shaft 28 is shown via the position of the drill pin 30 in the pin guide 58 of the input shaft 18. The drill pin 30 has moved proximally within the pin guide 58 (to its proximal end 41) as compared to its positioning in the first retracted configuration. In the second retracted configuration, the dog gear 40 has moved proximally, engaging the spur gear 38. As shown in FIG. 8, a feature 43 (e.g., flange) of the dog gear 40 engages a feature 45 (e.g., flange) of the spur gear 38. Due to the coupling of the dog gear 40 with the spur gear 38, the spur gear 38 begins to rotate with the rotation of the drill drive shaft 28 (via the input shaft 18). The rotation of the spur gear 38 of the drill drive shaft 28 is translated to the spur gear 50 on the inserter drive shaft 42, causing the rotation of the inserter drive shaft 42 through the square nut (or hub) 44 and within the second drive shaft recesses 26.

As also shown in FIG. 8, the spur gear 50 comprises the square drive 52 and the inserter drive shaft 42 comprises the distal square portion 35, allowing for the transfer of rotation from the spur gear 50 to the inserter drive shaft 42. Thus, at the second (or fully) retracted configuration, the inserter drive shaft 42 begins to translate distally in the second drive shaft recesses 26. The translation is permitted due to threads 37 at the proximal end 46 of the inserter drive shaft 42. In other words, the square nut 44 remains in place, while the inserter drive shaft 42 translates distally via rotation of the threads 37 within and through the square nut 44. As the inserter drive shaft 42 rotates, the threads 37 push the anchor driver (or inserter) 200 out through the second guide tube 56. Throughout this process, the user is maintaining an input of rotation from a handpiece or other power device (e.g.; drill) on the input shall 18.

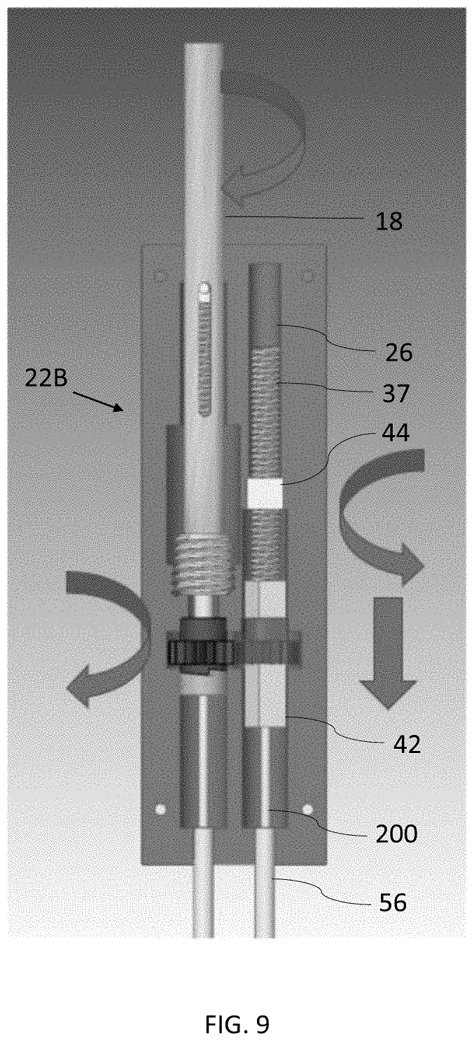

FIG. 9 shows the anchor system 10 in a first insertion configuration, according to an embodiment. From the second retracted configuration, the user continues to input rotation to the input shaft 18 and the inserter drive shaft 42 continues to translate distally in the second drive shall recesses 26 through the square nut 44. In the first insertion configuration shown in FIG. 9, the inserter drive shaft 42 is rotating at the same pitch of the threads 37 thereon.

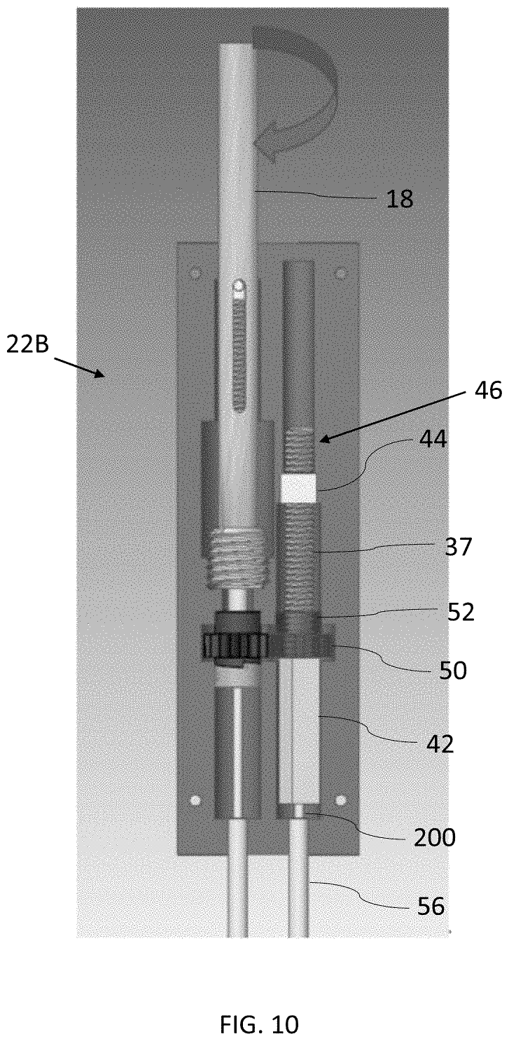

FIG. 10 shows the anchor system 10 in a second insertion configuration, according to an embodiment. In the second insertion configuration, an anchor (not shown) on the anchor driver (or inserter) 200 has reached the desired insertion depth in the pilot hole (not shown), Once the desired insertion depth is achieved, the inserter drive shaft 42 is disengaged from the square drive 52 of the spur gear 50, as shown. The inserter drive shaft 42 is held at the desired insertion depth due to the engagement of the threads 37 on the proximal end 46 of the inserter drive shaft 42 with the square nut 44.

In the second insertion configuration, no matter how much rotation is applied to the input shall 18, no additional translation is provided. This eliminates the risk of continually driving or rotating the anchor into the pilot hole even though the desired or predetermined depth has been reached. From this point, an indicator (not shown) on the inserter drive shaft 42 or within the second drive shaft recess 26 will advise the user that the insertion is complete. The indicator can vary in position on the anchor system 10 based on the type of anchor deployed. The user will remove the entire anchor system 10 from the positioned location with the anchor inserted in the pilot hole.

The automated anchor insertion system 10 ultimately increases user efficiency. It ensures that the anchor is inserted into the pilot hole created. It gives the user the ability to focus just on the location of the anchor instead of handling multiple devices. It also eliminates the risk of continually driving the anchor into the pilot hole past the desired depth. Most importantly, the anchor system 10 is a platform that can be used with both soil and rigid anchors.

While embodiments of the present invention has been particularly shown and described with reference to certain exemplary embodiments, it will be understood by one skilled in the art that various changes in detail may be effected therein without departing from the spirit and scope of the invention as defined by claims that can be supported by the written description and drawings. Further, where exemplary embodiments are described with reference to a certain number of elements it will be understood that the exemplary embodiments can be practiced utilizing either less than or more than the certain number of elements.

What is claimed is:

1. An automated anchor insertion system, comprising:
a body having a first end and a second end;
an input shaft extending from the first end of the body and a guide tube extending from the second end of the body;
a first drive shaft recess within the body;
a drill drive shaft moveable within the input shaft; and
wherein in a first configuration, the input shaft and the drill drive shaft move distally together through the first drive shaft recess and in a second configuration, the drill drive shaft moves proximally relative to the input shaft.

2. The system of claim 1, further comprising a gear connected to a distal end of the input shaft, wherein in the second configuration, the gear mates with threads in the first drive shaft recess.

3. The system of claim 1, further comprising a pin guide extending through the input shaft.

4. The system of claim 3, further comprising a feature of the drill drive shaft which is moveable within the pin guide.

5. An automated anchor insertion system, comprising:
a body having a first end and a second end;
an input shaft extending from the first end of the body and a guide tube extending from the second end of the body;
a first drive shaft recess and a second drive shaft recess within the body;
a drill drive shaft moveable within the input shaft;
wherein in a first configuration, the input shaft and the drill drive shaft move distally together through the first drive shaft recess and in a second configuration, the drill drive shaft moves proximally relative to the input shaft;
an inserter drive shaft moveable within the second drive shaft recess; and
wherein in a third configuration, movement of the drill drive shaft moves the inserter drive shaft.

6. The system of claim 5, wherein in the third configuration, the drill drive shaft is connected to a first gear and the inserter drive shaft extends through a second gear, and rotation of the first gear rotates the second gear.

7. The system of claim 6, wherein in a fourth configuration, the inserter drive shaft is not within the second gear and does not move in response to rotation of the first gear.

8. The system of claim 6, further comprising a feature connected to a distal end of the drill drive shaft which is configured to connect the drill drive shaft to the first gear.

9. The system of claim 6, wherein the inserter drive shaft has a distal square portion which is sized and configured to extend through a square drive of the second gear and threads at a proximal end.

10. The system of claim 5, further comprising a pin guide extending through the input shaft, wherein a feature of the drill drive shaft is moveable within the pin guide.

11. The system of claim 5, wherein the guide tube is bifurcated, having a first guide tube extending into the first drive shaft recess and a second guide tube extending into the second drive shaft recess.

12. A method for drilling a pilot hole and inserting an anchor, comprising the steps of:
providing a body having a first end and a second end, an input shaft extending from the first end of the body and a guide tube extending from the second end of the body, a first drive shaft recess and a second drive shaft recess within the body, a drill drive shaft moveable within the input shaft and connected to a drill bit, and an inserter drive shaft moveable within the second drive shaft recess and connected to an anchor driver;
driving the input shaft, which drives the input shaft and the drill drive shaft together in a distal direction and extends the drill bit through the guide tube, drilling the pilot hole;
connecting the input shaft to the first drive shaft recess; and
retracting the drill bit by driving the input shaft and moving the drill drive shaft independently in a proximal direction.

13. The method of claim 12, further comprising the steps of connecting a feature at a distal end of the drill drive shaft to a first gear within the first drive shaft recess, wherein the first gear engages a second gear in the second drive shaft recess, the second gear having the inserter drive shaft extending therethrough.

14. The method of claim 13, further comprising the step of rotating the second gear, causing the inserter drive shaft to rotate through the second gear and move the anchor driver distally through the guide tube.

15. The method of claim 14, further comprising the step of continuing rotation of the second gear until the inserter drive shaft extends entirely through and past the second gear.
